(19) United States
(12) Patent Application Publication
(10) Pub. No.: US 2009/0062710 A1
(43) Pub. Date: Mar. 5, 2009

(54) SOLID AND PAINLESS STICKING BAR

(76) Inventor: Yu-Ho LIN, Nei-Pu Hsiang (TW)

Correspondence Address: SINORICA, LLC, 528 FALLSGROVE DRIVE, ROCKVILLE, MD, 20850, US

(21) Appl. No.: 11/849,290

(22) Filed: Sep. 2, 2007

(51) Int. Cl. A61F 15/00

(52) U.S. Cl. 602/42

(57) ABSTRACT

A solid and painless sticking bar includes a solid main body and an adhesive member fixed at the underside of the main body. The main body has plural cut lines respectively provided at a proper interval so that the main body can be cut into a proper length or height for use according to practical needs. Plural main bodies are protuberantly fixed at proper locations around the outer circumferential edge of a wound by means of the adhesive member. Thus, when bandaging material is covered or wound on the main bodies and positioned over the wound, it will not directly contact with the surface of the wound and adhere to the wound when the wound secretes mucus, able to prevent the wound from being injured for a second time when the bandaging material is removed from the wound.

### BACKGROUND OF THE INVENTION

1. Field of the Invention

This invention relates to a solid and painless sticking bar, particularly to one to be protuberantly fixed at an outer circumferential edge of a wound to prevent bandaging material from directly contacting with the surface of the wound and avoid the wound being injured for a second time when the bandaging material is removed from the wound.

2. Description of the Prior Art

Generally, when a person's skin is scraped and injured, the wound of the skin is first applied with ointment for eliminating inflammation and healing the wound and then the wound is covered or wound up with gauze or permeable bandage to prevent the wound from directly touched or infected by germs. However, the gauze is usually contacted directly with the wound and covered on the whole wound in a multilayer winding way, resulting in bad permeability at the location of the wound. Although permeable bandage has its surface formed with numerous permeation holes, yet the permeable bandage, due to its water-resisting property, must have a certain extent of viscosity for adhering to a man's skin, and thus the permeable bandage may unexpectedly pull and drag the skin around the wound and touch the wound. In addition, when the wound tissue secretes mucus, the gauze or the permeable bandage is easy to adhere to the wound; therefore, when the gauze or the permeable bandage is removed from the wound, the wound way be ripped and injured for a second time.

### SUMMARY OF THE INVENTION

The objective of this invention is to offer a solid and painless sticking bar able to be optionally cut into a proper size to tally with the range of a wound. The solid and painless sticking bars are protuberantly fixed around the outer circumferential edge of the wound to prevent gauze or bandage from directly touching and adhering to the surface of the wound when the wound secretes mucus.

The solid and painless sticking bar in the present invention includes a solid main body and an adhesive member fixed at the underside of the main body. The main body is rectangle-shaped, polygon-shaped or a cylinder-shaped, having plural cut lines respectively provided at a proper interval. The main body can be cut into a proper length or height according to practical needs to be fixed at a proper location of the outer circumference of a wound. The adhesive member disposed at the underside of the main body is double-sided glue, an adhesive agent or adhesive material harmless to a man's skin for positioning the main body to prevent it from falling off.

By so designing, the solid and painless sticking bar protuberantly fixed at the outer circumferential edge of a wound can function to prevent bandaging material (gauze or permeable bandage) from directly contacting with the wound during carrying out bandaging, able to avoid the wound being injured for a second time when the gauze or the bandage is removed from the wound.

### BRIEF DESCRIPTION OF DRAWINGS

This invention will be better understood by referring to the accompanying drawings, wherein:

FIG. 1 is a perspective view of a solid and painless sticking bar in the present invention;

FIG. 2 is a perspective view of the solid and painless sticking bar to be used in the present invention;

FIG. 3 is a perspective view of the solid and painless sticking bar in a first using condition in the present invention; and

FIG. 4 is a perspective view of the solid and painless sticking bar in a second using condition in the present invention.

### DETAILED DESCRIPTION OF THE PREFERRED EMBODIMENT

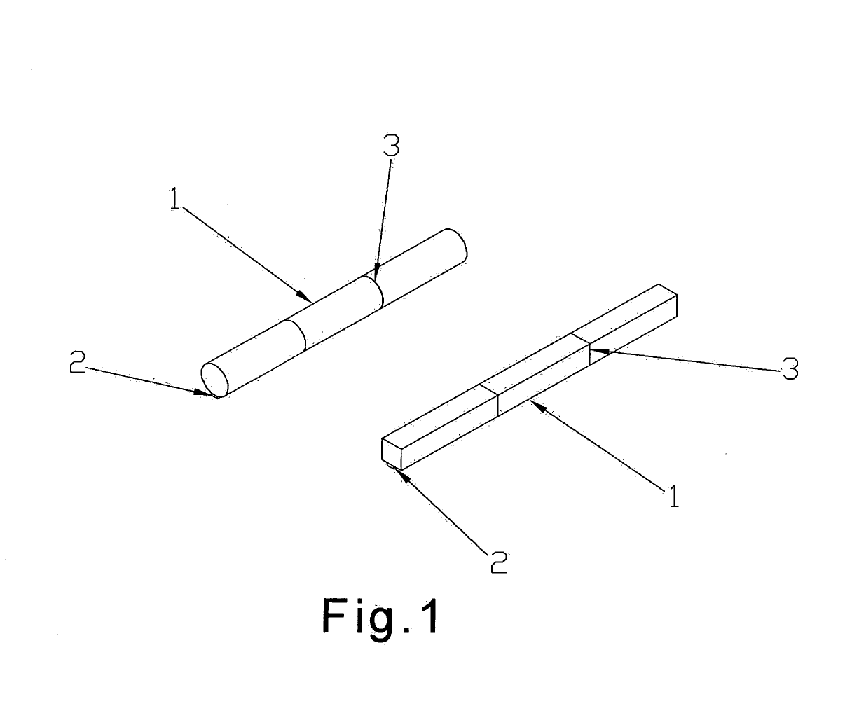

A preferred embodiment of a solid and painless sticking bar in the present invention, as shown in FIG. 1, includes a solid main body 1 and an adhesive member 2 combined together.

The main body a is rectangle-shaped, polygon-shaped or cylinder-shaped, having plural cut-off lines 3 respectively provided at a proper interval for facilitating cutting the main body 1 into a proper length or height for use.

The adhesive member 2 is double-sided glue, adhesive agent or adhesive material harmless to a man's skin, positioned at the underside of the main body 1 for fixing the main body 1 in position to prevent it from falling off.

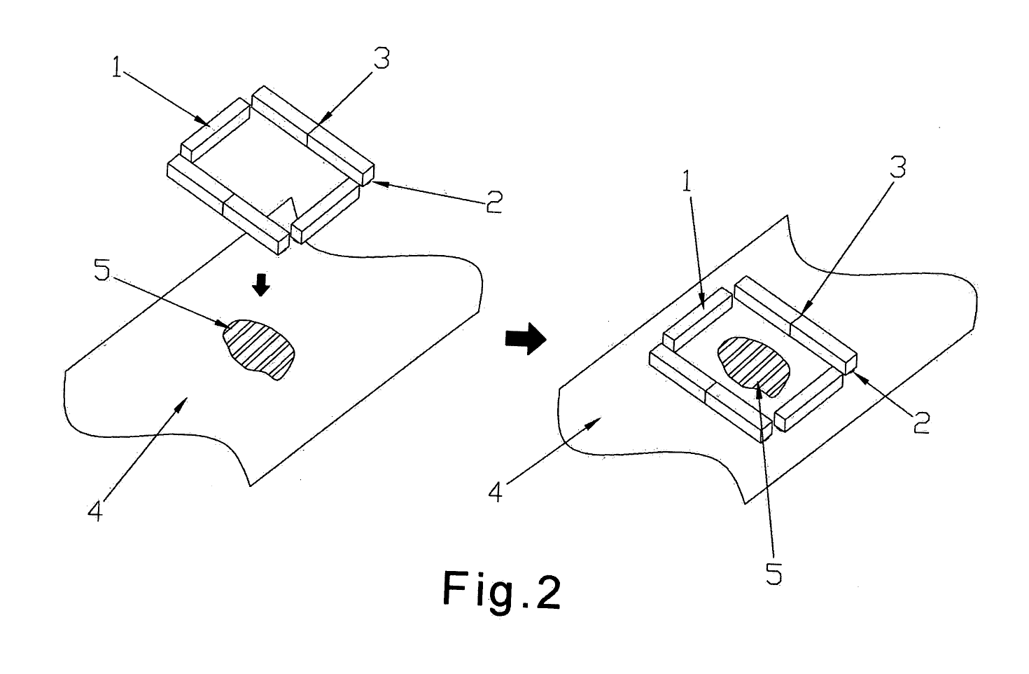

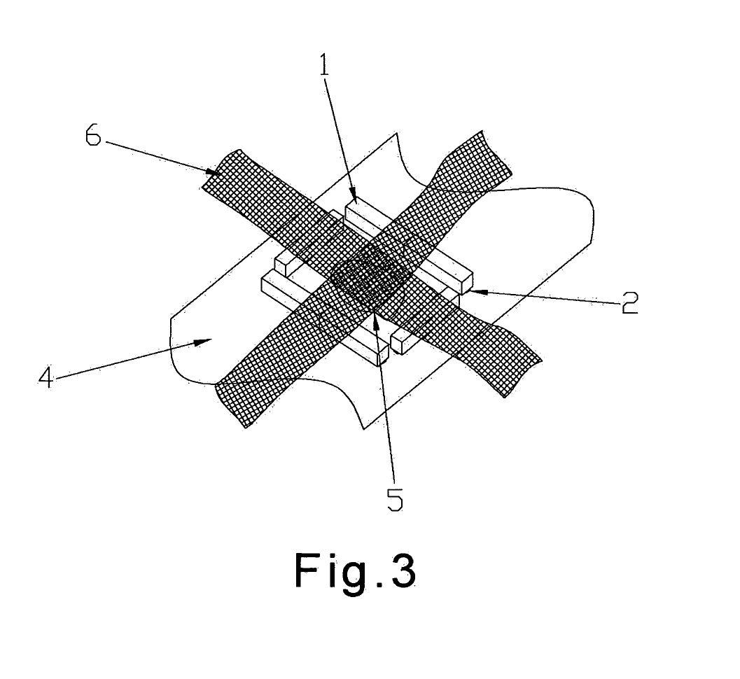

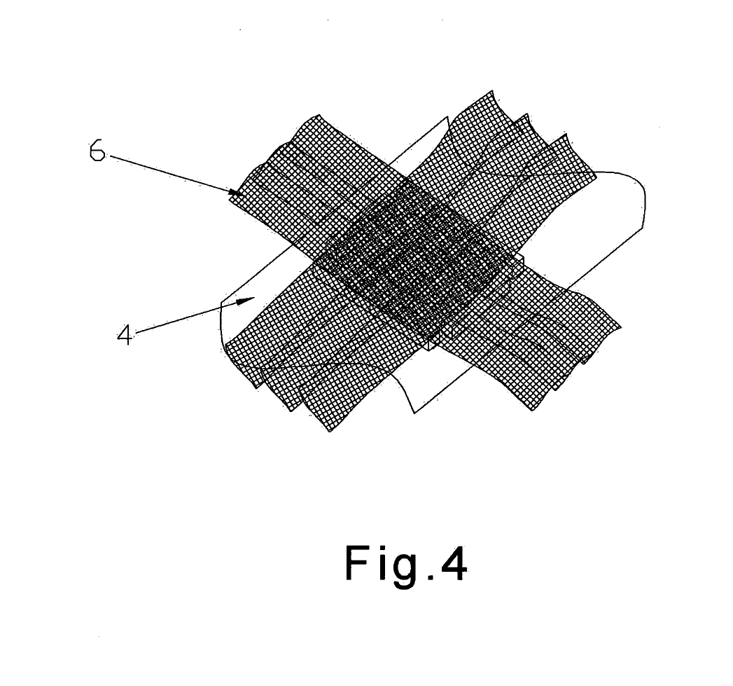

In using, referring to FIGS. 2, 3 and 4, firstly, estimate the range of a wound 5, and then measure and cut off the main body 1 at one of the cut lines 3 to obtain a proper length or height needed for tallying with the range of the wound 5. Subsequently, the main bodies 1 are fixed at proper locations of the outer circumference of the wound 5 of a man's skin 4 by means of the adhesive member 2 under each of the main bodies 1, letting the main bodies 1 protuberantly positioned around the outer circumference of the wound 5. Lastly, the bandaging material 6, gauze or permeable bandage, is covered or wound on the main bodies 1 and positioned over the wound 5, finishing treatment of the wound 5.

Specifically, the plural solid main bodies 1 are protuberantly positioned at proper locations of the outer circumferential edge of the wound 5 for propping up the bandaging material 6 to prevent the bandaging material 6 from directly contacting with the surface of the wound. Therefore, when the wound 5 tissue secretes mucus, the bandaging material 6 can be avoided adhering to the wound 5, and the wound 5 can be prevented from being ripped and injured for a second time when the bandaging material 6 is removed from the wound 5.

While the preferred embodiment of the invention has been described above, it will be recognized and understood that various modifications mat be made therein and the appended claims are intended to cover all such modifications that may fall within the spirit and scope of the invention.

1. A solid and painless sticking bar comprising a solid main body, said main body having cut-off lines respectively provided at a proper interval, said solid main body fixed at a proper location at an outer circumferential edge of a wound, said solid main body cut into a proper length or height for use according to practical needs.

2. The solid and painless sticking bar as claimed in claim 1, wherein an adhesive member is disposed at an underside of said solid main body for fixing said solid main body in position.

3. The solid and painless sticking bar as claimed in claim 1, wherein said solid main body is shaped as rectangular.

4. The solid and painless sticking bar as claimed in claim 1, wherein said solid main body is polygon-shaped.

5. The solid and painless sticking bar as claimed in claim 1, wherein said solid main body is cylinder-shaped.

6. The solid and painless sticking bar as claimed in claim 2, wherein said adhesive member is double-sided glue.

7. The solid and painless sticking bar as claimed in claim 2, wherein said adhesive member is an adhesive agent.

8. The solid and painless sticking bar as claimed in claim 2, wherein said adhesive member is adhesive material harmless to a man's skin.
